(19) United States
(12) Patent Application Publication
(10) Pub. No.: US 2006/0045849 A1
(43) Pub. Date: Mar. 2, 2006

(54) Method of customizing the fragrance of nasal medications

(76) Inventor: Farhan Taghizadeh, Uniontown, PA (US)

Correspondence Address: Michael L. Weiner;Biomed Solutions LLC, Suite 215, 150 Lucius Gordon Drive, West Henrietta, NY, 14586, US

(21) Appl. No.: 10/924,489

(22) Filed: Aug. 24, 2004

(51) Int. Cl. A61L 9/04

(52) U.S. Cl. 424/45

(57) ABSTRACT

Disclosed is a process for improving the scent of nasal medication comprising the steps of presenting a person with a fragrance choice comprised of a plurality of fragrance selections, receiving from the person a selection of a fragrance from said fragrance choice, thereby producing a selected fragrance, and adding to a nasal medication a scenting agent wherein the scenting agent causes a fragrance of the nasal medication to substantially mimic the selected fragrance, thereby producing a scented nasal medication.

### FIELD OF THE INVENTION

This invention generally relates to the field of fragrant nasal medications, and in particular to a process for the production thereof.

### BACKGROUND OF THE INVENTION

Topical nasal medications are a mainstay of treating nasal diseases. Examples of such sprays include topical isotonic, hypertonic, and hypotonic nasal saline sprays, wet preparation nasal steroid sprays, antihistamine sprays, mast cell stabilizer sprays, anti-vasomotor rhinitis sprays like ipratropium, decongestant sprays, and the use of nebulized antibiotics. Topical nasal application are becoming a more popular pharmacological delivery method, with a variety of biologically active agents ranging from pain medication to estrogen to new cold vaccines being delivered through this format. A number of such devices are known to those skilled in the art.

U.S. Pat. No. 6,576,224 to Osbakken (Aersolized Anti-infectives, Anti-inflammatories, and Decongestants for the Treatment of Sinusitis) teaches various pharmaceutical compositions that are “formulated as a solution in a unit dose for aerosol administration to treat chronic sinusitis.”

U.S. Pat. No. 6,494,204 to Ponce (Nasal Medication Administering Device) teaches a nasal medication device “for providing a quick and easy way of safely administering medication to a user.”

U.S. Pat. No. 6,076,520 to Cooper (Device for Nasal Therapeutic Inhalation) provides “a nasal inhalation device for nebulizing liquid, including medication, using in the treatment of upper respiratory and other conditions . . . ”

U.S. Pat. No. 3,812,853 to Crain (Apparatus for Applying Medication or the like to Human Nasal Passages) discloses an “apparatus for applying a solution in particle form to a body cavity such as the nasal passage for medicinal, therapeutic and hygiene purposes.”

U.S. Pat. No. 6,419,901 to Placke (Method for Treating Neoplasms by Inhalation) teaches “a formulation, method and apparatus for treating neoplasms such as cancer by administering a pharmaceutically effective amount of highly toxic compositions by inhalation, wherein the composition is a nonencapsulated antineoplastic drug.”

U.S. Pat. No. 6,468,798 to Debs (Expression of Cloned Genes in the Lung by Aerosol and Liposome-Based Delivery) discloses gene therapy through the use of aerosols. Disclosed in the abstract of this patent is “Methods and compositions for producing a mammal capable of expressing an exogenously supplied gene in cells of the airway are disclosed. Liposome-nucleic acid complexes are prepared then delivered via aerosol to the lung airway.”

U.S. Pat. No. 6,749,835 to Lipp (Formulation for Spray-Drying Large Porous Particles) teaches the advantages of aerosol medications. “Pulmonary delivery advantageously can reduce or eliminate the need for injection. For example, the requirement for daily insulin injections can be avoided. Furthermore, the particles of the invention can be delivered as a dry powder to the deep lung, upper or central airs. They can be used to provide controlled systemic or local delivery of therapeutic or diagnostic agents to the respiratory tract via aerosolization.”

The entire content of U.S. Pat. Nos. 3,812,853; 6,076,520; 6,419,901; 6,468,798; 6,494,204; 6,576,224; and 6,749,835 is hereby incorporated by reference into this specification.

The advantages of aerosol medications can be clearly seen by reference to the aforementioned patents. One of the major complaints of these medications is their fragrance; many of which are unpleasant. Some, like Flonase® have a flowery fragrance, while others lack any fragrance at all. The issue of product fragrance is believed to be one of the contributors to patient non-compliance. The issue of product fragrance seems to be exacerbated by certain preservatives like benzalkonium chloride (BKC), which prevents bacterial growth in medicated solutions. Other companies make medication fragrance a significant marketing factor in their un-fragranced products (for example, Rhinocort Aqua®). It is clear that the issue of fragrance is a key component to the marketing, design, and compliance of nasally delivered pharmaceuticals. In the field of palatable medications, flavorings have been used to overcome this deficiency.

For example, U.S. Pat. No. 5,480,674 discloses “a flavor composition for an oral electrolyte hydrate solution comprises ethyl esters, citrus oil concentrates, and sweet and fruity flavor components.” It is disclosed in the background of this patent that “the reluctance by infants and small children to take oral medications of any type that are distasteful is a well recognized and wide-spread problem among all pediatric medication manufacturers.” This patent then lists flavoring ingredients such as allyl butyrate, allyl caproate, allyl hexanoate, and/or allyl caprylate among other flavoring agents. The content of U.S. Pat. No. 5,480,674 is hereby incorporated by reference into this specification.

U.S. Pat. No. 6,372,259 to Kumar (Palatable, Sustained Release Drug Granules) teaches methods for manufacturing more palatable tablets for mastication. Similarly, the company Flavor X® has a method of doing business that involves the customized flavoring of prescriptions. Reference may be had to their web page at http://www.flavorx.com. Other businesses customize the scents of personal hygiene products. Reference may be had to http://www.bathjunkie.com. None of the above references teach or suggest the process of customizing the scenting of nasal medications. The entire content of U.S. Pat. No. 6,372,259 is hereby incorporated by reference into this specification.

The prior art is drawn to the improvement of the flavor of palatable medications. It is silent with regard to the improvement of the fragrance of nasal medications. The prior art thus fails to disclose or suggest improvements to the olfactory qualities of such medications.

It is an object of this invention to provide at least one of the following: a medication which has had its olfactory properties adjusted to improve its fragrance; an aerosolized medication which has had its olfactory properties improved; a process whereby an existing medication may be provided with improved olfactory properties.

### SUMMARY OF THE INVENTION

In accordance with the present invention, there is provided a process for improving the scent of nasal medication comprising the steps of presenting a person with a fragrance choice comprised of a plurality of fragrance selections, receiving from the person a selection of a fragrance from said fragrance choice, thereby producing a selected fragrance, and adding to a nasal medication a scenting agent wherein the scenting agent causes a fragrance of the nasal medication to substantially mimic the selected fragrance, thereby producing a scented nasal medication.

### BRIEF DESCRIPTION OF THE DRAWINGS

The invention will be described by reference to the following drawings, in which like numerals refer to like elements, and in which:

FIG. 1 is a flow diagram of one process of the invention;

FIG. 2 is a pictorial flow diagram of one embodiment of the invention;

FIG. 3 is a pictorial flow diagram of another embodiment of the invention;

FIG. 4 is a pictorial flow diagram of another embodiment of the invention; and

FIG. 5 is a pictorial flow diagram of yet another embodiment of the invention.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DESCRIPTION OF THE PREFERRED EMBODIMENTS

In describing the present invention, a variety of terms are used in the description. Standard terminology is widely used in the art of the chemistry of fragrances. For example, one may refer to Pybus D. and Sell C. The Chemistry of Fragrances, Royal Society of Chemistry, 1999. The term “fragrance” refers to the perceived odor of a substance. The fragrance may be a “single scent” wherein the fragrance is caused by a single scenting agent. Alternatively, the fragrance may be the result of a plurality of scents caused by a plurality of scenting agents. Some fragrances are easily associated with real-world objects. For example, one can identify certain fruits solely by their fragrances. Other fragrances, most commonly perfumes, have a unique fragrance which does not correlate to any other substance other than the scenting agent itself.

This patent application generally pertains to the user selected addition of scenting agents to nasal medications. These medications include, but are not limited to nasal antihistamines (for example, azelastine), nasal steroids (examples include beclomethonase, Budesonide®, flunisolide, fluticasone, mometasone, and triamcinalone), nasal decongestants (example phenylephrine, oxymetazoline), atrovent nasal spray, cromolyn sodium nasal spray, narcotic nasal sprays (Stadol®), the use of topical antifungal and antibioitic nebulized medications (reference may be had to http://www.sinuspharmacy.com/), the use of salt based nasal sprays, and the use of other pharmacologic agents in the nose. Recently, new vaccines like the flu vaccine have been given intra-nasally. Given this new class of vaccines, additional scenting would certainly add value to this class.

The prior art discloses the use of fragrances in combination with a variety of products, but fails to disclose the use of nasal medications. For example, U.S. Pat. No. 6,244,265 to Cronk (Adhesively Applied External Nasal Strips and Dilators Containing Medications and Fragrances) discloses non-aerosolized medications being used in combination with cosmetic fragrances. In the specification of this patent it is disclosed that “In order to improve the shelf-life and in-use olfactory effectiveness of such products, fragrance delivery mechanisms are used.” Similar disclosures are contained in U.S. Pat. No. 6,276,360 to Cronk (Medicated Nasal Dilator) and U.S. Pat. No. 6,550,474 to Anderson (Microencapsulated Fragrances and Methods of Coating Microcapsules). These references fail to teach or suggest the use of scented nasal medications. In addition, these references fail to teach the role of the customer or patient in making a custom fragrance selection.

U.S. Pat. No. 6,598,627 to Manzari (Apparatus Suitable for Preparing a Custom Personal Care Composition) which teaches an apparatus “for providing a customized personal care product to a consumer at a location, typically the point of sale . . . ” This patent fails to disclose or to suggest the use of nasal medications in conjunction with the apparatus.

U.S. Pat. No. 6,575,383 to Dobler (Presented and Custom Scented Card Insert) likewise discloses custom scenting certain consumer products, but fails to teach or suggest the use of custom scented nasal medications.

U.S. Pat. No. 5,626,155 to Saute (Method of Creating Fragrances in Situ) teaches “a method of producing a custom fragrance . . . comprising the steps of selecting a series of basic scents.” This reference likewise fails to teach or suggest the use of custom scented nasal medications.

The content of U.S. Pat. Nos. 6,244,265; 6,276,360; 6,550,474; 6,598,627; 6,575,383; and 5,626,155 is hereby incorporated by reference into this specification.

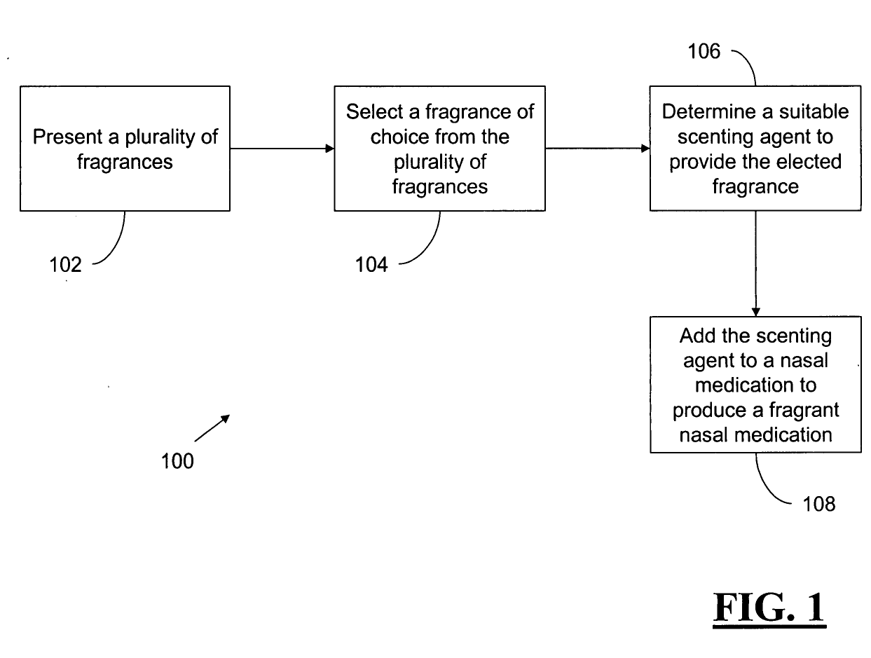

One embodiment is illustrated in FIG. 1 and process 100 depicted therein. In step 102 of process 100, a person is presented with a fragrance choice comprised of a plurality of fragrance selections. In step 104 of process 100, a fragrance is selected from the plurality of fragrances. For example, if cherry, apple, and banana fragrances are provided, the person may select the cherry fragrance. In step 106 of process 100, a suitable scenting agent is chosen so as to provide the elected fragrance. This scenting agent can be added to a nasal medication in step 108 of process 100 by the manufacturer, by the pharmacist, by the patient, or by a similar individual. Fragrances include, but are not limited to; almond, amariage, apple, banana, bay, rum, blackberry, camomille, chocolate, candy cane, cantaloupe, cedar, cherry, china lily, musk, cinnamon, citron, citrus, clove, coconut, columbian coffee, dewberry, eucalyptus, fig, frangipani, french vanilla, ginger, green, hyacinth, hibiscus, passion honeydew, honeysuckle, jasmine, key lime, kiwi, lavender, lemongrass, lemon, lilac, lily, mandarin orange, mango, mulberry, oatmeal, honey, patchouli, pear, peppermint, pineapple, kiwi pink, grapefruit, plumeria, peppermint, raspberry, rose, rosemary, sage, sweetgrass, cedar, spearmint, strawberry, sunflower, vanilla, waterlily, watermelon, and the like.

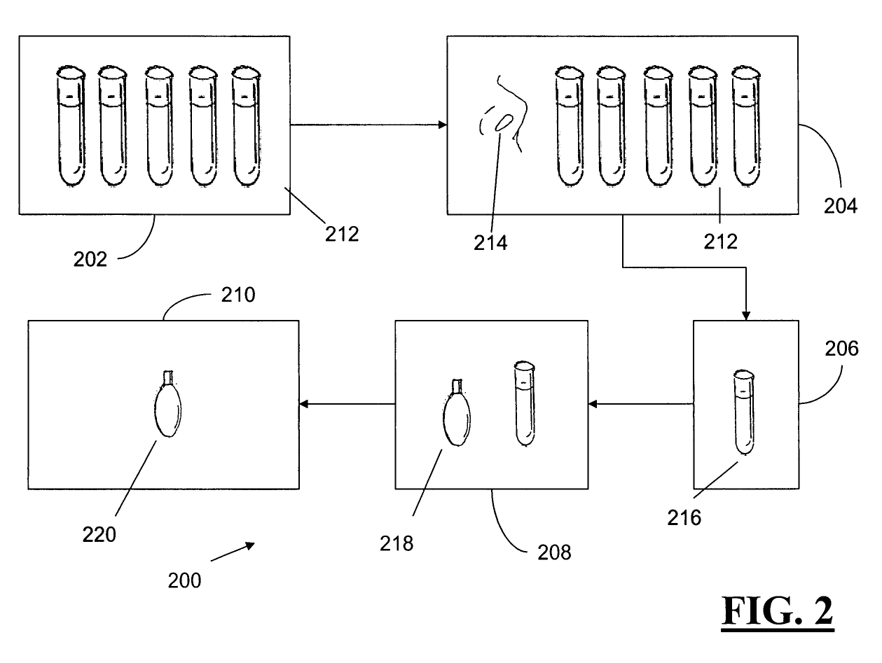

FIG. 2 depicts another embodiment in which the scenting agent itself is presented to the person. In process 200, and step 202 thereof, a plurality of fragrances 212 are presented to a person. In the embodiment depicted in FIG. 2, the fragrances are the scenting agents themselves. In step 204, the person 214 smells the plurality of fragrances 212 and selects a scenting agent. In step 206, a selected scenting agent 216 is taken from the plurality of fragrances. In step 208 the selected scenting agent 216 is mixed with nasal medication 218. In step 210, fragrant nasal medication 220 is produced from the mixing that occurred in step 208.

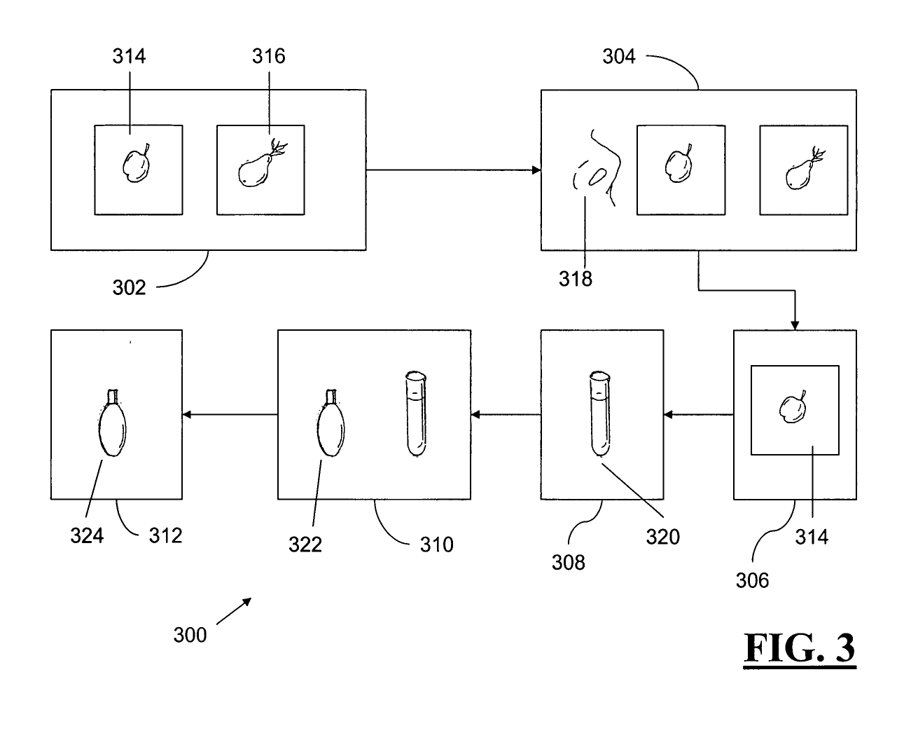

FIG. 3, and process 300 depicted therein, illustrates another embodiment of the instant invention. In process 300, an item is presented which has a fragrance substantially similar to the scenting agent. This second item is referred to as a “fragrance proxy.” For example, one may be presented with a card with a fragrance similar to vanilla, even though the card may not comprise the same scenting agent as will be added to the nasal medication. In the embodiment presented in FIG. 3, a plurality of fragrances is presented in step 302 wherein an apple scented card 314 and a pear scented card 316 are presented. In one embodiment cards 314 and 316 obtain their odor from the same chemical that will be used as the scenting agent. In another embodiment, the cards obtain their odor from a chemical that smells substantially similar to the odor of the end product, but wherein the chemical is different than the scenting agent to be used. In step 304, the person 318 smells cards 314 and 316 and selects a fragrance. In step 306, the fragrance selection is made. In the embodiment depicted, apple scented card 314 was selected. In step 308, a scenting agent 320 is chosen that will result in the end product having a fragrance substantially similar to scented card 314. In step 310, the scenting agent 320 is mixed with nasal medication 322. In step 312, this mixing results in fragrant nasal medication 324 which smells substantially similar to scented card 314.

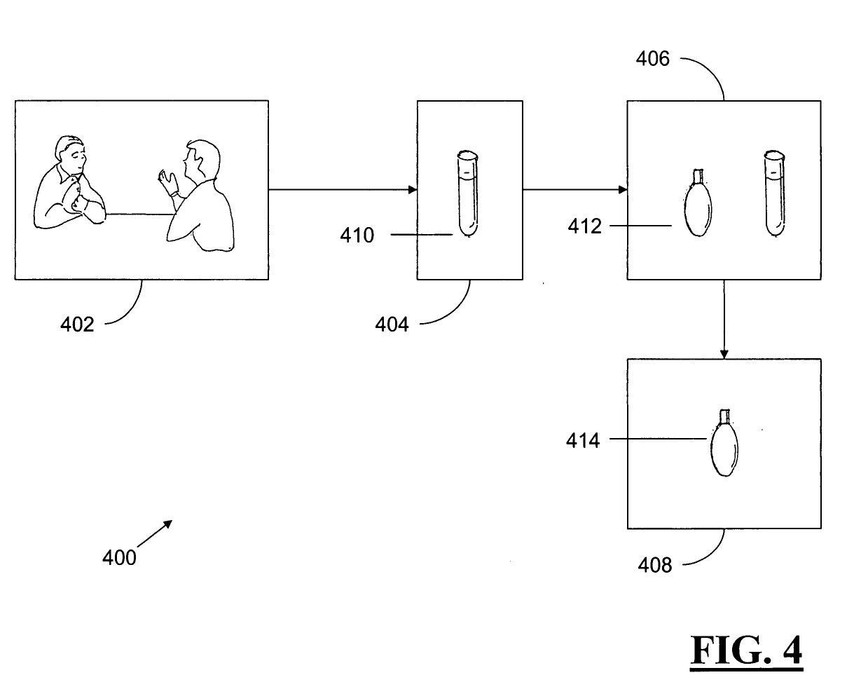

In the embodiment depicted in FIG. 4, and process 400 depicted therein, the person is presented with the scenting options using a “verbal cue,” rather than being physically exposed to the fragrance. In step 402, an oral description of the fragrance is presented to the person. In step 404, a scenting agent 410 is selected based on the conversation that took place in step 402. In step 406, the selected scenting agent 410 is mixed with nasal medication 412. In step 408 this mixing results in scented nasal medication 414.

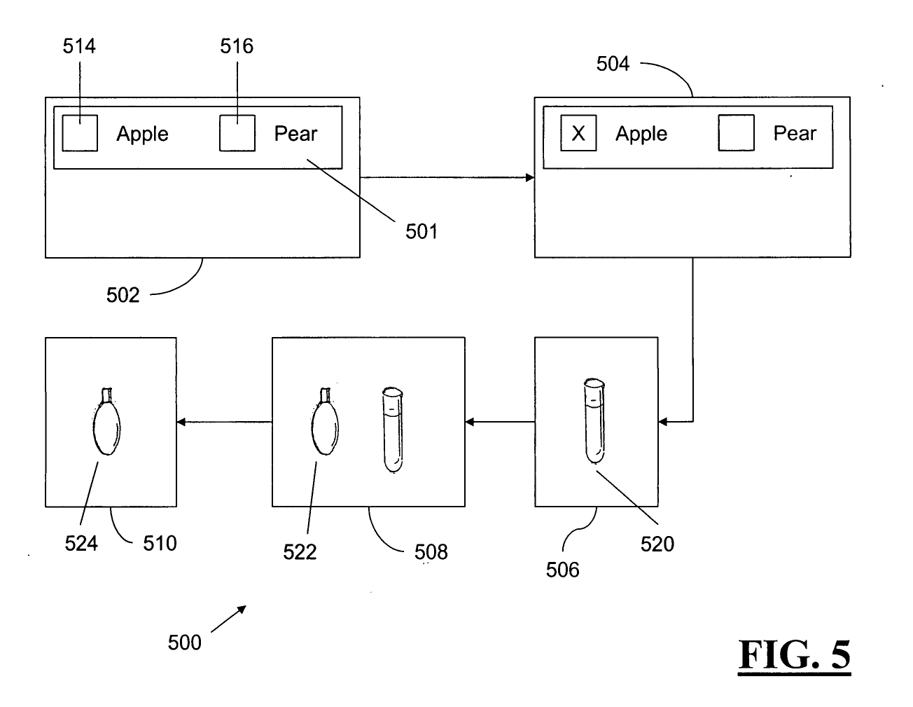

In the embodiment depicted in FIG. 5, the person is presented with the scenting options using a “visual cue.” In step 502 of process 500, card 501 is presented whereon the words “Apple” and “Pear” appear so as to function as a written description of the fragrance. In another embodiment (not shown), the visual cue is a picture that invokes a mental impression of the fragrance. For example, a picture of an apple or pear may be on the card. On card 501, check box 514 and 516 appear next to “Apple” and “Pear” respectively. In step 504, the person indicates their selection. In the embodiment depicted in FIG. 5, this indication is a written indication that takes the form of an “X” in check box 514. In another embodiment (not shown), the indication is verbal. In step 506 of process 500, a scenting agent 520 is chosen that corresponds to the selected fragrance. In step 508, this scenting agent is mixed with nasal medication 522. In step 510, this mixing results in fragrant nasal medication 524.

In another embodiment, a combination of the above techniques is utilized. For example, a combination of visual cues, verbal cues, and fragrance proxies are employed in the same presentation. In another embodiment, a person is presented with a fragrance choice by presenting visual cues over the internet.

Suitable scenting agents are known to those skilled in the art. For example, if the selected fragrance was vinegar, one may use acetic acid as a scenting agent. In another embodiment, the selected fragrance is bitter almond, and the scenting agent is benzaldehyde. In another embodiment, the selected fragrance is butter and the scenting agent is 2,3-butanone. In another embodiment, the selected fragrance is a fruity order, and the scenting agent is butanoic acid ethyl ester. In another embodiment, the selected fragrance is ethereal, and the scenting agent is 2-butanone. Other suitable fragrance and scenting agents include eucalyptus leaves (1,8-cineole), lemons (3,7-dimethyl-2,6-octadienal), roses ((E)-3,7-dimethyl-2,6-octadien-1-ol), apples (ethyl-2-methylbutanoate), almond (hydrogen cyanide), rotten eggs (hydrogen sulfide), vanilla (4-hydroxy-3-methoxybenzaldehyde), ripe raspberries (3-(4-hydroxyphenyl)-2-butanone), pear (isoamyl acetate), pineapple (isoamyl proprionate), cloves (2-methoxy-4-(2-propenyl)pheol), whiskey (3-methyl-1-butanol), fecal (3-methyl-1H-indole), peppermint (5-methyl-2-(1-methylethyl)cyclohexanone), woody (5-methyl-2-(1-methylethyl)phenol), skunk (2-methyl-2-propanethiol), spearmint (1-carvone), mandarin peel (d-carvone), rancid cheese (3-methylbutanoic acid), a sharp, pungent odor (2-methylpropanal), onion (3-(methylthio)propionaldehyde), rotten fish (n,n-dimethylmethanamine), cinnamon (3-phenyl-2-propenal), roses (2-phenylethanol), garlic (2-propene-1-sulfinothioic acid s-2-propenyl ester), fishy (pyridine), violets (4-(2,6,6-trimethyl-2-cyclohexen-1-yl)-3-buten-2-one and 4-(2,6,6-trimethyl-1-cyclohexen-1-yl)-3-buten-2-one), berry (ω-pentadecalactone), and the like. The above list is intended to be illustrative only. Other scenting agents are readily apparent to those skilled in the art. Similarly, a plurality of scenting agents may act together to provide an overall fragrance. The use of multiple scenting agents is common practice in the perfume industry. For example, if a first scenting agent is deemed to be too sweet, indole may be added as a second scenting agent to dull the fragrance. Many extracts use a plurality of scenting agents. For example, a rose extract is comprised of a plurality of scenting agent that provides the overall rose fragrance. It should also be clear that a scenting agent may be chosen that provides a fragrance that is unlike any other fragrance. For example, a fragrance may be “fruity”, yet not smell like any particular fruit.

Scenting agents generally act to impart a fragrance to a substance. Such scenting agents are distinguished from odor elimination compounds that attempt to remove odor. Examples of such odor elimination compounds include, for example, zeolites, activated charcoal, sodium bicarbonate, antimicrobial agents, and the like. Reference may be had to U.S. Pat. Nos. 5,885,559; 6,303,111; and the like.

The content of U.S. Pat. Nos. 5,885,559 and 6,303,111 are hereby incorporated by reference into this specification.

Numerous methods of adding scenting agents to substrates are known to those skilled in the art. In one embodiment, conventional means are used to combine the scenting agent and the nasal medication. In one such embodiment, the substrates are physically mixed. In another embodiment, the device of U.S. Pat. No. 6,598,627 is used. The content of U.S. Pat. No. 6,598,627 is hereby incorporated by reference into this specification.

In one aspect of the instant invention, a nasal medication has its fragrance modified. As used in this specification, the term “nasal medication” refers to a biologically active composition designed to be administered to the nasal passages of a biological organism that falls under the jurisdiction of the United States Food and Drug Administration. Reference may be had to Title 21 of the Code of Federal Regulations and the definitions referenced therein. In one embodiment, the nasal medication is a prescription nasal medication. In another embodiment, the nasal medication is a non-prescription nasal medication. In one embodiment, the nasal medication is an aerosolized nasal medication. In another embodiment, the nasal medication is not aerosolized. In yet another embodiment, the nasal medication is operatively configured to be aerosolized prior to use. Examples of nasal medications include nasal steroids, nasal antibiotics, nasal antivirals, nasal antifungals, nasal decongestants (oxymetazoline, phenylephrine) or others like atrovent (ipratropium bromide generic), nasal irrigants (generally a formulation for irrigating the nasal passageway or the sinuses; some examples include isotonic (such as Ocean Spray®) hypertonic, or hypotonic sprays, Nasal Vaccines (Influenzae, etc), Nasal Analgesics, Nasal Antimetabolites, Nasal Estrogen/Progesterone Hormone Preparations, and the like.

The prior art contains examples of medications which are fragrant. These fragrant medications, however, have not had their fragrances modified. Moreover, the fragrances were not selected from a plurality of fragrance selections presented to an end user. Examples of such fragrant medications include U.S. Pat. No. 6,060,045 to Mettler (Vitamized Air Freshener and Room Deodorizer Pad); U.S. Pat. No. 6,086,853 to Michaels (Medicated Vapor Candle); U.S. Pat. No. 6,238,646 to Zembrodt (Aqueous Aerosol Compositions for Delivery of Atomized Oil); and the like. The content of U.S. Pat. Nos. 6,060,045; 6,086,853; and 6,238,646 is hereby incorporated by reference into this specification.

In one embodiment of the present invention, a fragrance is added to a nasal medication through the use of a scenting agent. As used in this specification, the term “scenting agent” refers to a chemical composition that is perceived as having a particular fragrance. It is preferred that this scenting agent is substantially biologically inactive. In one embodiment, the scenting agent is comprised of a plurality of scenting substrates. In one such embodiment, a plurality of scenting substrates synergistically function together to generate the overall fragrance of the scenting agent. Reference may be had, for example, to U.S. Pat. No. 5,626,155 to Saute (Method of Creating Fragrances in Situ), the content of which is hereby incorporated by reference into this specification.

Scenting agents generally function so as to substantially mimic a selected fragrance. As used in this specification, the term “substantially mimic” refers to the ability of a scenting agent to approximate the scent of a selected sample. The level of mimicry is such that the casual observer is likely to be reminded of the fragrance of the selected sample to be mimicked.

In one embodiment, the nasal medication is the non-aerosolized saline solution disclosed in U.S. Pat. No. 6,669,059 to Mehta (System and Method for Passage Rinse). In another embodiment, the nasal medication is the non-aerosolized saline solution disclosed in U.S. Pat. No. 6,520,384 to Mehta (Apparatus and Method for Nasal Rinse). In yet another embodiment, the non-aerosolized nasal medication is delivered by means of the device disclosed in U.S. patent application 2002/0174864 to Alchas (Nasal Delivery Device Including Spray Nozzle). The content of U.S. Pat. Nos. 6,520,384; 6,669,059; and U.S. patent application 2002/0174864 is hereby incorporated by reference into this specification.

In another embodiment, the nasal medication is in particulate form and is delivered by means of the device disclosed in U.S. Pat. No. 3,812,853 to Crain (Apparatus for Applying Medication or the like to Human Nasal Passages). In another embodiment, the device is in powdered form and is delivered by means of the device disclosed in U.S. Pat. No. 4,227,522 to Carris (Inhalation Device). Numerous other methods for delivering nasal medications are known in the art. Reference may be had to U.S. Pat. No. 4,192,309 to Poulsen (Inhalation Device with Capsule Opener); U.S. Pat. No. 4,523,589 to Krauser (Method and Apparatus for Treating Ailments); U.S. Pat. No. 5,899,878 to Glassman (Nasal Irrigation System); U.S. Pat. No. 6,223,744 to Garon (Wearable Aerosol Delivery Apparatus); U.S. Pat. No. 6,505,662 to Py (System and Method for Application of Medicament into the Nasal Passage); and the like. The content of U.S. Pat. Nos. 3,812,853; 4,192,309; 4,227,522; 4,523,589; 5,899,878; 6,223,744; and 6,505,622 is hereby incorporated by reference into this specification.

It is therefore, apparent that there has been provided, in accordance with the present invention, a method for improving the scent of a nasal medication. While this invention has been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the appended claims.

I claim:

1. A process for improving the fragrance of nasal medication comprising the steps of:
a. presenting a person with a fragrance choice comprised of a plurality of fragrance selections,
b. receiving from said person a selection of a fragrance from said fragrance choice, thereby producing a selected fragrance, and
c. adding to a nasal medication a scenting agent wherein said scenting agent causes a fragrance of said nasal medication to substantially mimic said selected fragrance, thereby producing a scented nasal medication.

2. The process as recited in claim 1, wherein said nasal medication is a prescription nasal medication.

3. The process as recited in claim 1, wherein said nasal medication is aerosolized upon use.

4. The process as recited in claim 1, wherein said fragrance choice comprises a fragrance proxy.

5. The process as recited in claim 1, wherein said fragrance choice comprises a visual cue.

6. The process as recited in claim 1, wherein said fragrance choice comprises a verbal cue.

7. The process as recited in claim 1, wherein said fragrance choice comprises a sensory stimuli wherein said sensory stimuli is selected from the group consisting of a fragrance proxy, a visual cue, a verbal cue, and combinations thereof.

8. The process as recited in claim 1, wherein said nasal medication is a nasal antihistamine.

9. The process as recited in claim 1, wherein said nasal medication is a nasal steroid.

10. The process as recited in claim 1, wherein said nasal medication is a nasal decongestant.

11. The process as recited in claim 1, wherein said nasal medication is an atrovent nasal spray.

12. The process as recited in claim 1, wherein said nasal medication is a salt based nasal irrigation spray.

13. The process as recited in claim 1, wherein said nasal medication is a vaccine.

14. The process as recited in claim 1, wherein said fragrance is selected from the group consisting of almond, amariage, apple, banana, bay, rum, blackberry, camomille, chocolate, candy cane, cantaloupe, cedar, cherry, china lily, musk, cinnamon, citron, citrus, clove, coconut, Columbian coffee, dewberry, eucalyptus, fig, frangipani, french vanilla, ginger, green, apple, hyacinth, hibiscus, passion honeydew, honeysuckle, jasmine, key lime, kiwi, lavender, lemongrass, lemon, lilac, lily, mandarin orange, mango, mulberry, oatmeal, honey, patchouli, pear, peppermint, pineapple, kiwi pink, grapefruit, plumeria, peppermint, raspberry, rose, rosemary, sage, sweetgrass, cedar, spearmint, strawberry, sunflower, vanilla, waterlily, watermelon, and combinations thereof.

15. The process as recited in claim 1, wherein said person is selected from the group consisting of a manufacturer of said nasal medication, a pharmacist, and a patient.

16. A scented composition comprising
a. a nasal medication wherein said nasal medication has a first fragrance,
b. a scenting agent wherein said scenting agent has a second fragrance, wherein said scenting agent in combination with said nasal medication causes said scented composition to have a third fragrance.

17. The scented composition as recited in claim 16, wherein said third fragrance is substantially dissimilar to said first fragrance wherein said second fragrance was selected by a person from a plurality of fragrance selections.

18. The scented composition as recited in claim 17, wherein said person is selected from the group consisting of a manufacturer of said nasal medication, a pharmacist, and a patient.

19. The scented composition as recited in claim 16, wherein said nasal medication is selected from the group consisting of a nasal antihistamine, nasal steroid, nasal decongestant, an atrovent nasal spray, salt based nasal irrigation spray, and combinations thereof.

20. The scented composition as recited in claim 19, wherein said nasal medication is a prescription nasal medication.
